(19) United States
(12) Patent Application Publication
(10) Pub. No.: US 2026/0179025 A1
(43) Pub. Date: Jun. 25, 2026

(54) WHOLE-PROCESS TRACEABILITY SYSTEM FOR AQUATIC PRODUCTS BASED ON BLOCKCHAIN TECHNOLOGY AND IMPLEMENTATION METHOD THEREOF

(71) Applicant: South China Normal University, Guangzhou (CN)

(72) Inventors: Lihua ZENG, Guangzhou (CN); Yuning XUE, Guangzhou (CN); Juan FENG, Guangzhou (CN); Yihong CHEN, Guangzhou (CN); Xiaoying SONG, Guangzhou (CN); Chenhui ZHOU, Guangzhou (CN)

(73) Assignee: South China Normal University, Guangzhou (CN)

(21) Appl. No.: 19/536,670

(22) Filed: Feb. 11, 2026

(30) Foreign Application Priority Data
Jun. 12, 2025 (CN) 202510781701.1

(51) Int. Cl. G06Q 10/08; G06Q 10/087

(52) U.S. Cl. CPC G06Q 10/08; G06Q 10/087

(57) ABSTRACT

The present disclosure discloses a whole-process traceability system for aquatic products based on blockchain technology, wherein: The data acquisition layer is composed of environmental monitoring unit, feeding monitoring unit and RFID identification unit, which is used to collect aquaculture environmental data; the edge computing layer includes an edge server and an industrial control computer. The edge server and the industrial control computer preprocess the collected data; the blockchain layer is built on the Hyperledger Fabric platform to realize data tamper-proof storage, and the data authenticity is automatically verified through smart contracts. The application layer includes the aquaculture management subsystem, processing management subsystem, logistics management subsystem, and sales management subsystem, and adopts a multi-level authorization mechanism to manage data access rights. This method solves problems of data fraud, high traceability cost, and opaque information, and realizes the whole chain credible traceability of aquatic product quality and safety.

### TECHNICAL FIELD

The present disclosure relates to the field of quality control technology, in particular to a whole-process traceability system for aquatic products based on blockchain technology and an implementation method thereof.

### BACKGROUND

To strengthen the control over aquatic product quality and safety from the source and achieve informatized and digital management across the entire industrial chain, it is imperative to establish a traceability system covering all aspects, such as breeding, processing, warehousing, logistics, and sales. Traditional traceability methods, which mainly rely on centralized databases to store product information, face challenges such as data silos, susceptibility to tampering, and high traceability costs. The emergence of blockchain technology in recent years offers new solutions to these issues.

Blockchain is a decentralized distributed ledger technology. Utilizing cryptographic principles, it connects data in blocks and stores them across multiple nodes, enabling trusted data recording and consensus without centralized management. The characteristics of blockchain, including decentralization, tamper resistance, traceability, and high transparency, align well with the requirements for food safety traceability. Applying blockchain technology in the field of aquatic product quality and safety traceability is expected to address industry pain points such as information isolation, fraud reduction, and transparency improvement, thereby providing strong support for end-to-end quality control from pond to table.

Currently, both domestic and international efforts have begun to explore the application of blockchain technology in food traceability, with some achievements made. However, in the aquatic products sector, there remains a lack of mature, full-chain blockchain traceability solutions, particularly those integrated with smart devices and multi-scenario applications. Therefore, developing an intelligent blockchain-based traceability system suitable for all segments of the aquatic product industry chain is of great significance for ensuring product quality and safety and promoting the healthy development of the industry.

### SUMMARY

The purpose of the present disclosure is to provide a whole-process traceability system for aquatic products based on blockchain technology and an implementation method thereof to solve the problems of background technology.

In order to achieve the above purpose, the present disclosure provides a whole-process traceability system for aquatic products based on blockchain technology, including:
a data acquisition layer, including an environmental monitoring unit, a feeding monitoring unit, and an RFID identification unit, is configured to automatically collect aquaculture environmental data;
an edge computing layer, the edge computing layer includes an edge server and an industrial control computer, the edge server and the industrial control computer are configured to preprocess collected data;
a blockchain layer, built on a Hyperledger Fabric platform, is configured to achieve data tamper-proof storage and automatically verify data authenticity via smart contracts;
an application layer, the application layer includes an aquaculture management subsystem, a processing management subsystem, a logistics management subsystem, and a sales management subsystem, the application layer uses a multi-level authorization mechanism to manage data access rights.

In some embodiments, the environmental monitoring unit, feeding monitoring unit, and RFID identification unit are as follows:
The environmental monitoring unit includes a dissolved oxygen sensor, a pH sensor and a temperature sensor; the dissolved oxygen sensor, pH sensor and temperature sensor are all connected to the industrial control computer via a RS485 bus, each sensor adopts a stainless steel shell with a waterproof grade of IP67; a sensor probes is vertically installed at 30-50 cm below a water surface of an aquaculture pond;
the feeding monitoring unit includes a feeding quantity sensor and a feeding time recorder; the feeding quantity sensor is a weighing sensor with a range of 0-100 kg, installed at a bottom of the hopper of feeding equipment, and connected to the industrial control computer via the RS485 bus after being connected in series with the feeding time recorder;
the RFID identification unit includes an RFID reader and an RFID electronic tag; the RFID reader uses a high-frequency reader with a working frequency of 13.56 MHz and a reading distance of 0-10 cm, the RFID reader is connected to an industrial control computer through a TCP/IP protocol; the RFID electronic tag adopts a waterproof package and has a unique identification code for recording batch information of aquatic products.

In some embodiments, a specific structure of the feeding monitoring unit is as follows:
the feeding amount sensor adopts a parallel beam structure, a main material is 304 stainless steel, a sensitivity is 2 mV/V±0.1 %, an accuracy level is C3, a temperature compensation circuit is placed inside, a working temperature range is −20° C. to +60° C., the temperature compensation circuit is fixed at the bottom of the hopper by four M12 bolts, an inclination angle is not more than 2°;
the feeding time recorder uses an industrial-grade single-chip microcomputer controller with a main frequency of 100 MHz, a real-time clock chip, a time accuracy of ±1 s/day, a 3.5-inch LCD, a protection level IP65, and a 24V DC power supply, a UPS backup power supply can work continuously for 4 hours and is placed inside;
a 4-core shielded cable is used to connect the feeding quantity sensor and the feeding time recorder; an outer diameter of the cable is 8 mm and a length of the cable is not more than 5 meters; a connection terminal adopts a crimping terminal, and a signal line adopts a twisted-pair structure to suppress a common mode interference; the RS485 bus uses a T-type three-way connector to realize a daisy chain connection, a 120Ω terminal resistor is installed at an terminal of the bus, and a communication rate is set to 9600 bps.

In some embodiments, the edge server and industrial control computer are as follows:
the industrial control computer uses a Linux real-time operating system to collect sensor data through a data acquisition program; the data acquisition program includes a data verification module, a data filtering module, and a data compression module; the data verification module uses a CRC verification algorithm to ensure data integrity; the data filtering module uses a median filtering algorithm to remove outliers, and the data compression module uses a lossless compression algorithm to reduce an amount of data transmission;
the edge server adopts Docker containerized deployment and runs an edge computing framework, the edge computing framework includes a data analysis engine, a feature extraction engine and a data fusion engine; the data analysis engine parses collected original data into a standard format, the feature extraction engine uses a sliding time window method to extract data features, and the data fusion engine uses a Kalman filter algorithm to fuse multi-source data;
the edge server interacts with the blockchain layer through a message queue, and uses a publish/subscribe mode to realize an asynchronous data transmission, the message queue is implemented by RabbitMQ, and a data transmission security is ensured by SSL encryption.

In some embodiments, a specific implementation architecture of the industrial control computer is as follows:
the Linux real-time operating system uses an RTLinux kernel, configured for a real-time priority scheduling strategy SCHED_FIFO, and an interrupt response time is less than 100 microseconds; the system adopts a dual-partition structure, where a system partition is mounted in a read-only manner, and a data partition uses a journaling file system to ensure data integrity;
the data acquisition program is based on a multi-threaded architecture design, including a device driver thread, a data acquisition thread, and a data processing thread:
the device driver thread is responsible for managing a communication interface between an RS-485 device and a TCP/IP device; an epoll mechanism is used to achieve high-concurrent IO processing and support concurrent access of up to 1000 devices;
the data acquisition thread adopts a polling mode to sample each sensor at regular intervals, a sampling period can be configured in a range of 100 ms-1000 ms, and a double buffer mechanism is used to avoid data loss;
the data processing thread receives the collected data through a pipeline mechanism, and uses a zero-copy technology to improve data processing efficiency, the processed data interacts with an application program through shared memory;
the industrial control computer also includes a watchdog program, which is responsible for monitoring a running state of the system; when a system anomaly is detected, a relevant process is automatically restarted, meanwhile, a system log is recorded through a syslog mechanism to support a unified management of a remote log server.

In some embodiments, the Hyperledger Fabric platform adopts a consortium blockchain architecture, including a sorting node cluster, a peer node cluster and a channel management module; the sorting node cluster uses a Raft consensus algorithm to maintain a consistency of a blockchain ledger, the peer node cluster is responsible for maintaining a chain code and state database, and the channel management module is responsible for managing data isolation between different organizations;
the specific implementation architecture of the Hyperledger Fabric platform is as follows:
the sorting node cluster includes a master node and several slave nodes, and the nodes communicate with each other through a Raft consensus protocol:
the master node is responsible for receiving a transaction request and sorting according to a timestamp, a batch processing mechanism is used to package transactions into blocks, and a block size threshold is set to 2 MB or 500 transactions; the slave nodes are synchronized with the master node through a heartbeat mechanism, and a heartbeat interval is 100 ms, when the master node is detected to fail, an election mechanism is automatically triggered; a gRPC protocol is used to communicate between the nodes, and a TLS 1.3 encryption channel is used to support the concurrent operation of up to 5 nodes;
the peer node cluster adopts a hierarchical architecture design:
a consensus layer is responsible for verifying transactions and maintaining ledgers, LevelDB is used to store transaction data and supports up to 10 TB data storage, a chain code layer is responsible for executing smart contracts, using Docker container to isolate a running environment, and supporting smart contracts written by Golang, Java and Node. js; a state database layer is implemented by CouchDB, which supports JSON format data storage and rich query functions, and a data synchronization delay does not exceed 100 ms;
the channel management module adopts policy-based access control:
the channel configuration adopts MSP (member service provider) to manage identity and supports X.509 certificate authentication, a channel strategy is defined by a signature policy language, which supports AND, OR, NOutOf, and other combinational logic, data isolation is based on a multi-ledger mechanism, and each channel maintains independent ledger data;
the smart contract includes data verification contract, storage contract and access control contract; the data verification contract adopts digital signature algorithm and timestamp verification mechanism to ensure a credibility of data source; the storage contract adopts Merkel tree structure and IPFS distributed storage to realize data tamper resistance, the access control contract realizes fine-grained rights management based on ABAC (attribute-based access control) model;
it also includes a blockchain network management module, which is responsible for node certificate management, blockchain network monitoring, and performance optimization, where a certificate management adopts PKI architecture, network monitoring is implemented by Prometheus, and performance optimization is achieved by dynamically adjusting block size and transaction concurrency.

In some embodiments, the aquaculture management subsystem, processing management subsystem, logistics management subsystem, and sales management subsystem are as follows:
the aquaculture management subsystem adopts B/S architecture, including an aquaculture environment monitoring module, a feed delivery management module and a growth cycle management module; the aquaculture environment monitoring module uses WebSocket to push environmental data in real time, the feed delivery management module realizes intelligent feeding quantity prediction based on deep learning algorithm, and the growth cycle management module uses GIS technology to realize visual management of aquaculture ponds;
the processing management subsystem and the logistics management subsystem adopt micro-service architectures and are implemented through a Spring Cloud framework; the processing management subsystem includes a processing procedure tracking module, a quality inspection module and an inventory management module; the logistics management subsystem includes a transportation monitoring module, a temperature monitoring module and a distribution management module; a data interaction between each module is performed through RESTful API;
the sales management subsystem integrates a mobile payment interface and a traceability code query function, adopts an OAuth2.0 protocol to realize user authentication, and verifies product traceability information through blockchain smart contract; the multi-level authorization mechanism is based on the RBAC (role-based access control) model, including four privilege levels of system administrator, enterprise administrator, operator and consumer; different privilege levels correspond to different data access scopes and operation privileges.

In some embodiments, the aquaculture environment monitoring module, feed delivery management module, and growth cycle management module are as follows:
the aquaculture environment monitoring module adopts a hierarchical architecture design:
the data acquisition layer implements real-time data push through a WebSocket server, the heartbeat detection mechanism is adopted to maintain a connection state, and supports up to 1000 concurrent connections; a data display layer adopts an ECharts chart library to realize data visualization, supports multi-index linkage display of temperature, pH value, dissolved oxygen, etc., and a refresh frequency can be configured for 1-60 seconds; an alarm management layer uses a rule engine to realize a threshold alarm, and supports multi-channel push alarm information such as SMS, mail and WeChat;
the feed delivery management module is designed based on a deep learning framework:
the data preprocessing layer uses a sliding time window for data segmentation, a window size is 24 hours and a step size is 1 hour; the model training layer adopts the LSTM neural network structure, including three layers of LSTM units, 128 neurons in each layer, and a dropout rate is 0.5; a predictive analysis layer uses an Adam optimizer with a learning rate of 0.001, and predicts an optimal feeding amount based on historical feeding data and environmental parameters;
the growth cycle management module is based on GIS technology:
the map engine layer adopts an OpenLayers framework to support pond boundary drawing and spatial analysis, a data management layer adopts a PostGIS extension to realize spatial data storage, supporting latitude and longitude and plane coordinate system; a business function layer integrates a visual display of attribute information such as pond number, breeding variety, and stocking density.

In some embodiments, a specific implementation architecture of the sales management subsystem is as follows:
a mobile payment interface adopts a unified payment gateway design:
a payment gateway layer integrates a SDK of mainstream payment channels such as Alipay and WeChat payment, and adopts a adapter mode to realize a unified interface; a transaction processing layer adopts a distributed transaction framework TCC mode to ensure an atomicity of the payment process; a reconciliation and liquidation layer adopts a regular task mode, and a transaction reconciliation is performed at 2 a.m. every day to support automatic error checking.

a traceability code query function is based on QR code technology:
The QR code generation layer adopts a QR Code coding standard, and an error correction level is Level Q, which supports 2048 bytes of information storage, a data coding layer adopts Base64 coding format, including product ID, production batch, time stamp and other information; a analytical verification layer invokes a block link port through a REST API to verify an authenticity and effectiveness of the code;
OAuth2.0 authentication adopts a layered design:
an authentication service layer supports a password mode and an authorization code mode; an access token is valid for 2 hours, and a refresh token is valid for 30 days; a resource service layer adopts a JWT format token and uses a RSA-256 algorithm to sign; a user management layer is docked with the blockchain smart contract to realize the chain storage of user identity information.

An implementation method of the whole-process traceability system for aquatic products based on blockchain technology, including the following steps:
S1, collecting multi-source heterogeneous data such as water quality parameters, feeding records, and breeding batches in a breeding process automatically through the equipment of the environmental monitoring unit, feeding monitoring unit, and RFID identification unit to realize an automation of data collection, and an automation rate of collection is more than 95 %;
S2, using the industrial control computer as an edge gateway of data acquisition, performing a preprocessing of checking, filtering, compression etc., for original collected data; the edge server further processes the data, such as data cleaning, feature extraction, multi-source fusion, etc., while improving the data quality, it minimizes an amount of data transmission and reduces a storage and computing burden of a blockchain;
S3, uploading optimized structured data to a blockchain network for storage through an API interface provided by a smart contract according to a unified data format, so as to realize traceability and non-tampering of a whole life cycle of the data;
S4, combined with cutting-edge technologies such as big data analysis and artificial intelligence, each business application subsystem (including breeding, processing, warehousing, logistics and other links) calls the blockchain data to perform real-time monitoring, intelligent early warning and process optimization of production and operation activities, so as to improve a refinement level of quality management and an efficiency of supply chain coordination;
S5, management personnel can manage a background through a Web, consumers can enter a product traceability code through a mobile APP and other terminals to query quality and safety information of product production, processing, warehousing, transportation, and other links in real time, thereby enabling the complete visualization and control of the product quality process.

Therefore, the present disclosure adopts the above-mentioned whole-process traceability system of aquatic products based on blockchain technology and an implementation method thereof, which has the following beneficial effects:
(1) Blockchain technology has been introduced into the field of aquatic product quality and safety traceability. By leveraging the characteristics of blockchain, such as decentralization, tamper resistance, traceability, and high transparency, it addresses from the root the pain points of the traditional traceability system, including data fraud, high traceability costs, and information opacity. Furthermore, it enables information sharing and business collaboration across all links in the aquatic product supply chain.
(2) By integrating RFID and other automatic identification technologies with IoT sensing devices, the automatic collection of key quality data during the breeding process has been achieved, with an automation rate of up to 95%. By assigning electronic “identity cards” to aquatic products, digital archives covering their entire lifecycle have been established, significantly enhancing the efficiency and accuracy of traceability.
(3) The concept of edge computing has been introduced to implement data layering. By deploying edge gateways and edge servers at the data generation source to process data locally and extract key features, it not only reduces data transmission pressure but also alleviates the storage and computational load on the blockchain. This makes the blockchain system lighter, greatly improving its robustness and scalability.
(4) The application layer incorporates cutting-edge technologies such as big data and artificial intelligence. Through in-depth analysis of on-chain data and data-driven intelligent decision-making, it achieves refined and intelligent production management. Particularly in the breeding process, data modeling is used to optimize feeding strategies and provide early warnings for epidemic disease risks. This makes breeding more scientific and standardized, significantly improving breeding efficiency and yield.
(5) The introduction of blockchain and smart contracts offers a new solution for identity authentication and rights management of each participant in the supply chain. Based on smart contracts and a fine-grained authorization mechanism, it not only effectively protects business secrets but also promotes cross-organizational data sharing and collaboration. This reflects a seamless integration of centralized control and decentralized incentives.

The following is a further detailed description of the technical scheme of the present disclosure through drawings and implementation examples.

### BRIEF DESCRIPTION OF THE DRAWINGS

FIG. 1 is a schematic diagram of the system architecture of the present disclosure;

FIG. 2 is a structural diagram of the RFID identification unit of the present disclosure;

FIG. 3 is a data processing flow chart of the edge computing layer of the present disclosure;

FIG. 4 is a schematic diagram of the blockchain layer architecture of the present disclosure;

FIG. 5 is a flow chart of the realization method of the present disclosure.

### DETAILED DESCRIPTION OF THE EMBODIMENTS

The following detailed description of the embodiment of the present disclosure provided in the accompanying diagram is not intended to limit the scope of the present disclosure requiring protection, but merely indicates the selected embodiment of the present disclosure. Based on the embodiments in this present disclosure, all other embodiments obtained by ordinary technicians in this field without making creative labor belong to the scope of protection of this present disclosure.

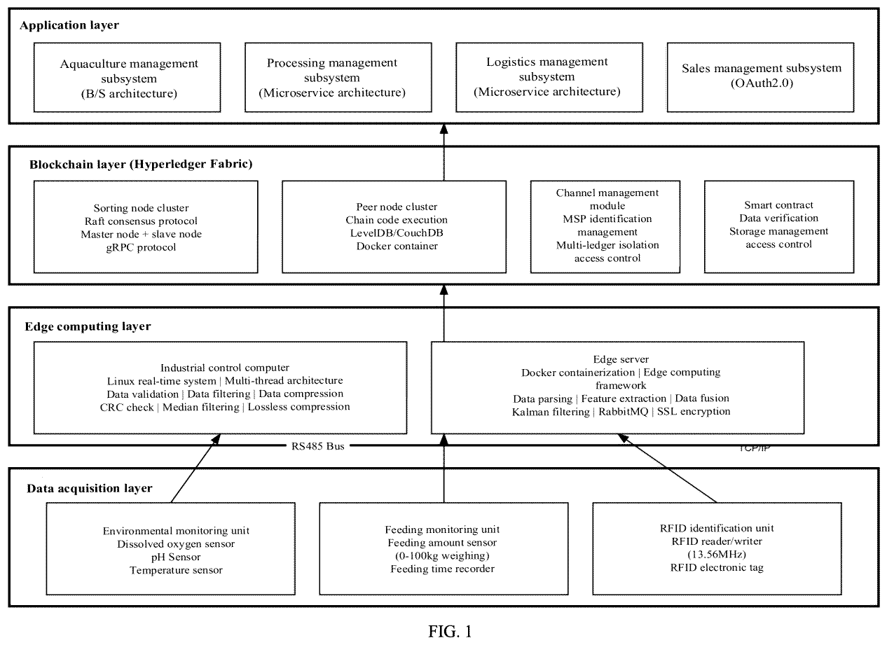

Please refer to FIG. 1, a whole-process traceability system for aquatic products based on blockchain technology, including a bottom-up hierarchical design of a data acquisition layer, an edge computing layer, a blockchain layer, and an application layer.

Among them, the data acquisition layer at the bottom is mainly responsible for the automatic collection and digitization of data. This layer includes an environmental monitoring unit, a feeding monitoring unit, and an RFID identification unit.

In the aquaculture stage, the environmental monitoring unit collects aquaculture environmental parameters, such as water temperature, pH, and dissolved oxygen, in real time via various sensors deployed in the aquaculture pond. As shown in FIG. 1, the unit primarily includes a temperature sensor, a pH sensor, and a dissolved oxygen sensor. Each sensor is connected to the industrial control computer via an RS485 bus. The sensor shell is constructed from IP67-grade stainless steel, which exhibits strong corrosion resistance. The probe portion is vertically suspended 30-50 cm below the water surface to avoid measurement deviations caused by sediment accumulation.

Simultaneously, the feeding monitoring unit is employed to record feeding data during the breeding process. As shown in FIG. 1, this unit includes a weighing sensor with a range of 0-100 kg, installed at the outlet of the feeding device's hopper to collect the weight of each feed portion in real time. It also incorporates a feeding time recorder, connected in series with the weighing sensor. Both devices transmit the collected data to the industrial control computer via the RS485 bus, thereby recording the time and quantity of each feeding event.

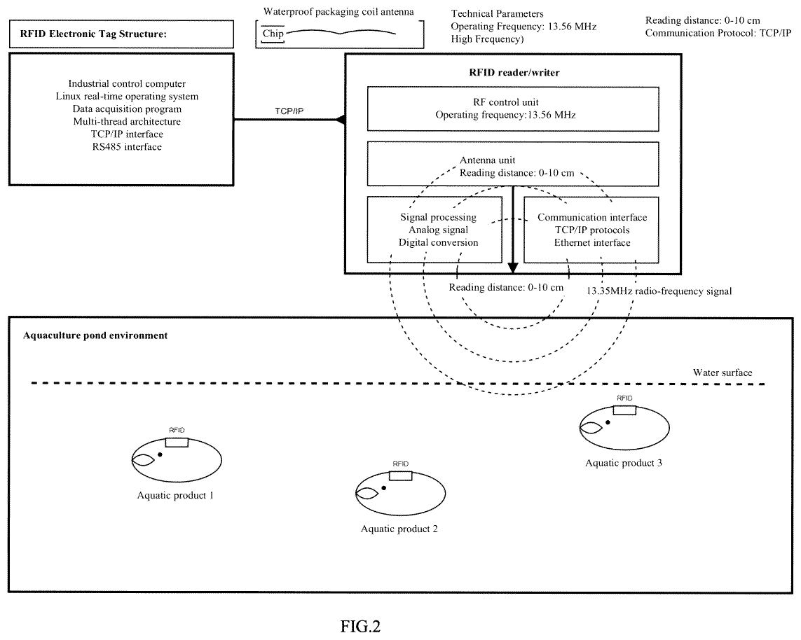

For the identification of aquatic products, this embodiment utilizes radio frequency identification (RFID) technology. As shown in FIG. 2, the RFID identification unit includes an RFID reader and an RFID electronic tag. The RFID reader operates at a high frequency of 13.56 MHz, provides a reading distance of up to 10 cm, and is connected to the industrial control computer via the TCP/IP protocol.

Each aquatic product is affixed with an RFID electronic tag. The electronic tag is packaged in a waterproof enclosure and contains a built-in unique identification code, which is used to record information such as the aquaculture batch and stocking date of the aquatic product. During the aquaculture process, when an aquatic product passes within range of the RFID reader, the reader automatically reads the electronic tag information and transmits it to the industrial control computer. This enables automatic identification of the aquatic product and supports digital management of aquaculture records.

The data acquisition layer submits the collected multi-source heterogeneous data to the edge computing layer of the previous layer for preprocessing. As shown in FIG. 1, the edge computing layer is mainly composed of the industrial control computer and edge server deployed in the farm area.

Among them, the industrial control computer plays the role of a data acquisition gateway. It uses a Linux real-time operating system to sample each sensor node regularly through polling, and the sampling period can be configured in the range of 100-1000 ms according to requirements. Considering the large volume of the original collected data, in order to improve the data quality and transmission efficiency, the data acquisition program of the machine is designed with modules such as data verification, data filtering, and data compression.

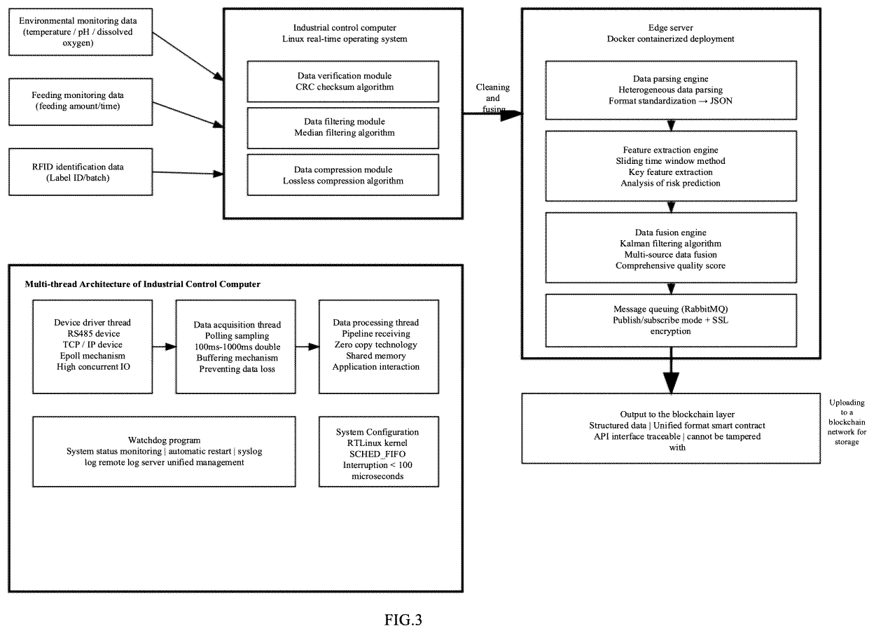

As shown in FIG. 3, the data verification module uses the cyclic redundancy check (CRC) algorithm to verify the collected data frames, eliminate the transmission error data, and ensure the integrity of the data; the data filtering module uses the median filtering algorithm to remove the dirty data and outliers caused by equipment failure or external interference, and improve the accuracy of the data. The data compression module uses a lossless compression algorithm (such as run-length coding) to compress the amount of data as much as possible and reduce the bandwidth usage of data transmission without affecting the accuracy of the data.

On the other hand, the edge computing layer also deploys several edge servers. As shown in FIG. 1, the edge server is deployed using the current popular Docker containerization technology and runs a self-developed edge computing framework. The framework further aggregates and fuses multi-source data, mines key features, sublimates data value, and provides a unified data entry for chain storage on the blockchain.

As shown in FIG. 3, the core of the framework includes a data parsing engine, a feature extraction engine, and a data fusion engine. Among them, the data analysis engine is responsible for semantic analysis and format conversion of heterogeneous data (such as environmental monitoring data, feeding monitoring data, RFID identification data, etc.) collected by the industrial control computer, and outputs unified JSON format data.

On this basis, the feature extraction engine uses a variety of data mining algorithms to extract key features that are important for the quality and safety risk prediction of aquatic products. For example, the sliding time window method is used to determine whether there is a risk of water quality deterioration in the past 24 hours; for another example, correlation analysis is used to judge the correlation strength between feeding amount and water quality, so as to provide a reference for intelligent feeding decisions.

The function of the data fusion engine is to use a multi-sensor fusion algorithm to fuse multi-source data to form a more comprehensive and reliable environmental portrait. For example, the Kalman filtering algorithm is used to fuse the measured values of temperature, pH value, dissolved oxygen, and other parameters to obtain the comprehensive quality score of the water body, which provides decision-making reference for the optimization of the aquaculture process.

In addition, in order to support real-time processing and reliable delivery of data, the edge server and the blockchain layer are loosely coupled through a message queue middleware. The middleware uses the current mainstream RabbitMQ, adopts the publish/subscribe mode, and a persistence mechanism to ensure that the data can be finally delivered in unexpected situations, such as network interruption. Meanwhile, the transmission channel uses SSL encryption to prevent data from being tampered with.

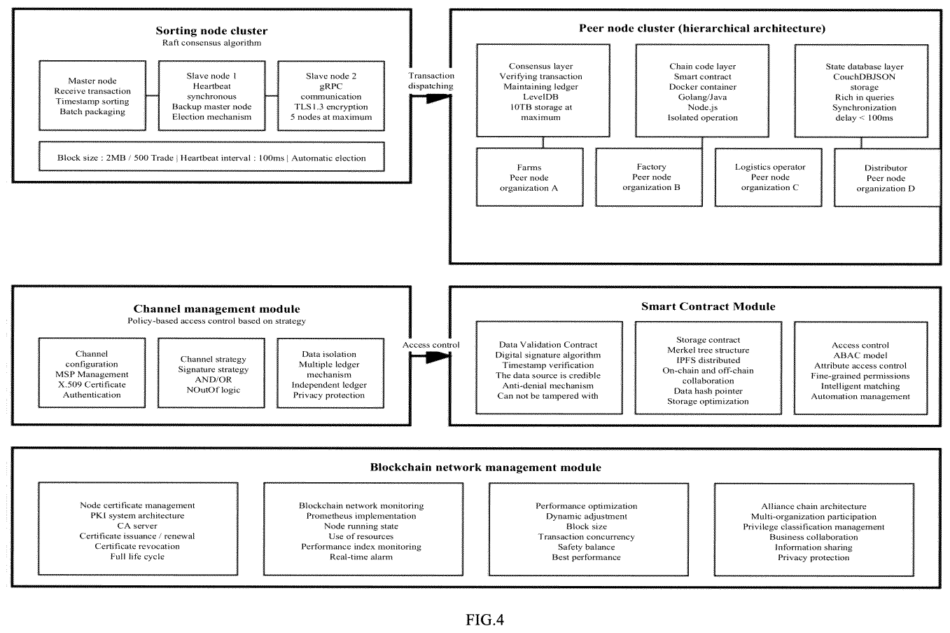

After the data is filtered and optimized layer by layer at the edge computing layer, it proceeds to the blockchain layer for on-chain storage. As shown in FIG. 4, the blockchain layer is structured as a consortium blockchain and is built using the highly mature Hyperledger Fabric blockchain platform.

From an architectural perspective, the blockchain network primarily consists of an ordering node cluster, a peer node cluster, and a channel management system. Among these, peer nodes are responsible for executing smart contracts (chaincode) and maintaining ledger data. Multiple peer nodes may form a cluster, representing an organization (such as farms, processing plants, distributors, etc.) to join the blockchain network.

The ordering node serves as a key hub of the entire network, responsible for receiving transaction requests, ordering them, packaging them into blocks, and then distributing the blocks to each peer node for verification, thereby achieving global consensus across the network. Ordering nodes can also form a cluster, using the Raft protocol to automatically elect a master node while the remaining nodes serve as slaves. The master and slave nodes remain synchronized in real time through a heartbeat mechanism. In the event of a master node failure, a slave node can immediately take over, ensuring high availability of the ordering service.

The channel management system is used to achieve data isolation. As participants in the aquatic product traceability system often require strong confidentiality for commercial data, it is not feasible for all members to have access to all data. Therefore, the platform establishes different channels according to permission levels, with each channel corresponding to a separate ledger. Only members within a given channel can access the data in its ledger, thereby protecting the privacy of all parties while still enabling data sharing.

The core functionalities of the blockchain are implemented through a series of smart contracts deployed on peer nodes. For example, the data verification contract employs an asymmetric encryption system to sign the data, combines it with a timestamp mechanism to generate a data digest, and uploads it to the blockchain network for storage, thereby ensuring that once data is on the chain, it cannot be repudiated or tampered with. The storage contract saves the complete data object into the InterPlanetary File System (IPFS) in object storage form, while storing the corresponding data hash as a pointer on the blockchain, achieving collaboration between on-chain and off-chain storage and solving the blockchain's inherent storage limitation. The access control contract is based on a fine-grained Attribute-Based Access Control (ABAC) model, which intelligently matches access policies according to the requester's identity attributes and data attributes, enabling automated privilege management. These foundational contracts can be flexibly assembled according to requirements to form complex business logic.

On this basis, the blockchain management system provides a series of supporting services, including member management, network monitoring, and performance optimization. Among them, the member management is based on PKI, and the CA server issues digital certificates for each node, and manages the whole life cycle of certificate application, renewal, revocation, etc., thus realizing trusted identity authentication. The network monitoring is based on DevOps tools such as Prometheus. The administrator can view the running status and resource usage of each node in real time. In terms of performance optimization, the best balance between blockchain security and performance can be sought by adjusting parameters such as block size and transaction concurrency.

The blockchain layer can be called by the application layer after the collected and fused data is trusted and stored. As shown in FIG. 1, the application layer includes different business subsystems for each link of production management, including the aquaculture management system, processing management system, logistics management system, and sales management system.

The aquaculture management system adopts the browser/server (B/S) architecture to provide information-based breeding services for farmers. For example, the environmental monitoring module can display the water quality parameters of the pond in real time, when a parameter exceeds the standard, the system automatically warns and links the knowledge base to prompt the best practices of pond management; the feeding management module is based on reinforcement learning and other algorithms, combined with the aquaculture environment and fish monitoring data, to learn and optimize the feeding strategy to achieve intelligent feeding. The growth cycle management module uses WebGIS to visualize the distribution of ponds, and visually displays the growth cycle of each batch of aquatic products from larvae to adult fish, which is convenient for overall scheduling.

The processing management, logistics management and other subsystems adopt the micro-service architecture and are developed based on the Spring Cloud framework. This architecture is easy to expand new functions, loosely coupled between services, and can be independently developed, tested, deployed, and scaled. For example, the processing management system includes micro-services such as process monitoring, quality and safety inspection, and warehouse inventory management, which are respectively connected to workshop MES, quality inspection system, WMS, etc. The logistics management system comprises micro-services such as capacity scheduling, temperature-control monitoring, and distribution tracking, which interface respectively with systems including a Transportation Management System (TMS), a cold-chain system, and express delivery providers. These micro-services interact with one another in JSON format via a RESTful API.

The terminal sales management system is mainly for consumers, providing product quality and safety information query services. Consumers only need to scan the QR code on the product packaging with a mobile phone to query the full chain information of the product from breeding to sales, and verify the authenticity of the product. The system uses the OAuth2.0 protocol for user authentication and authorization, and adapts to a variety of login methods (such as mobile phone number, micro-signal, face recognition, etc.) to enhance the user experience. In addition, the system is also embedded in the mobile payment SDK, which supports convenient payment methods such as code scanning payment and one-click payment, and realizes the closed-loop of online sales.

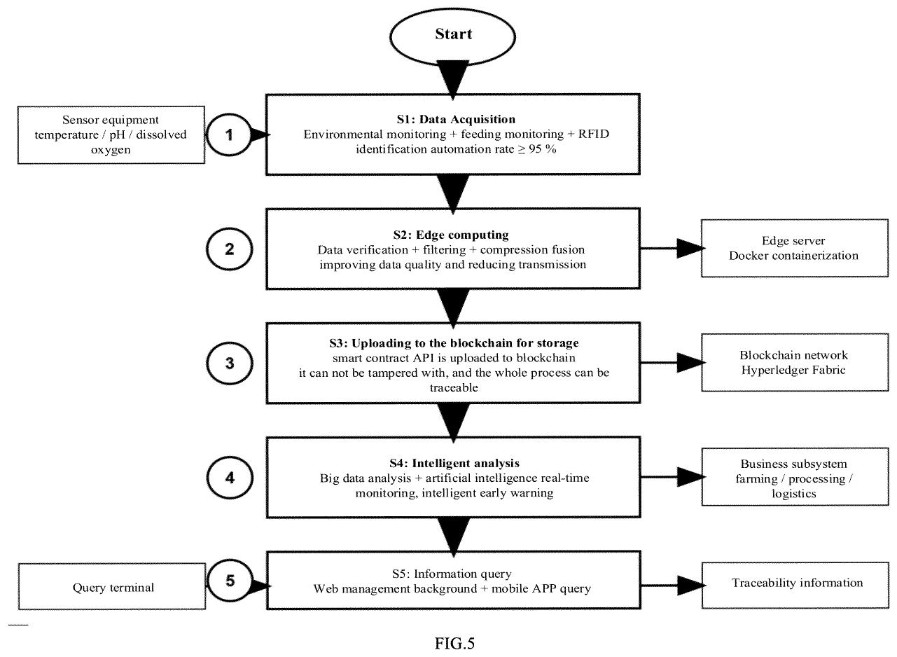

As shown in FIG. 5, an implementation method of the whole-process traceability system of aquatic products based on blockchain technology is also provided, including the following steps:
Step 1: Data collection, through the equipment of the environmental monitoring unit, feeding monitoring unit, and RFID identification unit, multi-source heterogeneous data, such as water quality parameters, feeding records, and breeding batches in the breeding process, are automatically collected to realize the automation of data collection, the automation rate of collection is more than 95%.
Step 2: Edge computing, the industrial control computer is used as the edge gateway of data acquisition to perform preprocessing such as verification, filtering, and compression of the original collected data; the edge server further processes the data, such as cleaning, feature extraction, and multi-source fusion, while improving the data quality, it minimizes the amount of data transmission and reduces the storage and computing burden of the blockchain.
Step 3: On the chain certificate, the optimized structured data is uploaded to the blockchain network through the API interface provided by the smart contract according to the unified data format, so as to realize the traceability and non-tampering of the whole life cycle of the data.
Step 4: Intelligent analysis, each business application subsystem (including breeding, processing, warehousing, logistics and other links) calls blockchain data, combined with cutting-edge technologies such as big data analysis and artificial intelligence, to perform real-time monitoring, intelligent early warning and process optimization of production and operation activities, so as to improve the refinement level of quality management and the efficiency of supply chain coordination.

Step 5: Information query, managers can manage the background through the Web, consumers can enter the product traceability code to query the quality and safety information of product production, processing, warehousing, transportation, and other links in real time through mobile APP and other terminals, thereby enabling the complete visualization and control of the product quality process.

Therefore, the whole-process traceability system for aquatic products based on blockchain technology and an implementation method thereof, as described in the present disclosure, can be applied not only to the field of aquatic products but also extended to other agricultural products such as livestock, poultry, vegetables, and fruits. This present disclosure is of significant importance for enhancing the level of food safety management, safeguarding public dietary health, and promoting the development of agricultural modernization.

Finally, it should be noted that the above embodiments are provided solely to explain the technical solution of the present disclosure and should not be construed as limiting it. Although the present disclosure has been described in detail with reference to preferred embodiments, those skilled in the art will understand that modifications or equivalent substitutions to the technical solution of the present disclosure may still be made. Such modifications or equivalent substitutions shall not cause the amended technical solution to depart from the spirit and scope of the present disclosure as claimed.

What is claimed is:

1. A whole-process traceability system for aquatic products based on blockchain technology, comprising:
a data acquisition layer, comprising an environmental monitoring unit, a feeding monitoring unit, and an RFID identification unit, configured to automatically collect aquaculture environmental data;
an edge computing layer, wherein the edge computing layer comprises an edge server and an industrial control computer, and the edge server and the industrial control computer are configured to preprocess collected data; and,
a blockchain layer, built on a Hyperledger Fabric platform, configured to achieve data tamper-proof storage and automatically verify data authenticity via smart contracts;
wherein the Hyperledger Fabric platform adopts a consortium blockchain architecture, comprising a sorting node cluster, a peer node cluster and a channel management module; wherein the sorting node cluster uses a Raft consensus algorithm to maintain consistency of a blockchain ledger, the peer node cluster is responsible for maintaining a chain code and state database, and the channel management module is responsible for managing data isolation between different organizations;
wherein the specific implementation architecture of the Hyperledger Fabric platform is as follows:
the sorting node cluster comprises a master node and several slave nodes, and the nodes communicate with each other through a Raft consensus protocol:
the master node is responsible for receiving a transaction request and sorting according to a timestamp, a batch processing mechanism is used to package transactions into blocks, the slave nodes are synchronized with the master node through a heartbeat mechanism; and a gRPC protocol is used to communicate between the nodes;
the peer node cluster adopts a hierarchical architecture design, wherein:
a consensus layer is responsible for verifying transactions and maintaining ledgers, LevelDB is used to store transaction data, a chain code layer is responsible for executing smart contracts, using Docker containers to isolate a running environment; and a state database layer is implemented by CouchDB;
the channel management module adopts policy-based access control, wherein:
the channel configuration adopts MSP (member service provider) to manage identity; a channel strategy is defined by a signature policy language; data isolation is based on a multi-ledger mechanism, and each channel maintains independent ledger data;
the smart contract comprises a data verification contract, a storage contract, and an access control contract; the data verification contract adopts a digital signature algorithm and a timestamp verification mechanism; the storage contract adopts a Merkel tree structure and IPFS distributed storage; the access control contract realizes fine-grained rights management based on the ABAC (attribute-based access control) model;
wherein the smart contract also comprises a blockchain network management module, wherein the blockchain network management module is responsible for node certificate management, blockchain network monitoring and performance optimization, where a certificate management adopts PKI architecture, network monitoring is implemented by Prometheus, and performance optimization is achieved by dynamically adjusting block size and transaction concurrency;
an application layer, the application layer comprises an aquaculture management subsystem, a processing management subsystem, a logistics management subsystem, and a sales management subsystem, the application layer uses a multi-level authorization mechanism to manage data access rights;
the aquaculture management subsystem adopts B/S architecture, comprising an aquaculture environment monitoring module, a feed delivery management module, and a growth cycle management module;
the aquaculture environment monitoring module adopts a hierarchical architecture design, wherein:
the data acquisition layer implements real-time data push through a WebSocket server; the heartbeat detection mechanism is adopted to maintain a connection state; a data display layer adopts an ECharts chart library to realize data visualization; an alarm management layer uses a rule engine to realize a threshold alarm;
the feed delivery management module is designed based on a deep learning framework, wherein:
the data preprocessing layer uses a sliding time window for data segmentation, a window size is 24 hours and a step size is 1 hour; the model training layer adopts the LSTM neural network structure, comprising three layers of LSTM units, 128 neurons in each layer, and a dropout rate is 0.5; a predictive analysis layer uses an Adam optimizer with a learning rate of 0.001, and predicts an optimal feeding amount based on historical feeding data and environmental parameters; and
the growth cycle management module is based on GIS technology, wherein:
the map engine layer adopts an OpenLayers framework, a data management layer adopts a PostGIS extension to realize spatial data storage; a business function layer integrates a visual display of attribute information such as pond number, breeding variety, and stocking density.

2. The whole-process traceability system for aquatic products based on blockchain technology according to claim 1, wherein the environmental monitoring unit, feeding monitoring unit, and RFID identification unit are as follows:
the environmental monitoring unit comprises a dissolved oxygen sensor, a pH sensor, and a temperature sensor; wherein the dissolved oxygen sensor, pH sensor, and temperature sensor are all connected to the industrial control computer via an RS-485 bus, and each sensor probe is vertically installed at 30-50 cm below a water surface of an aquaculture pond;
the feeding monitoring unit comprises a feeding quantity sensor and a feeding time recorder;
wherein the feeding quantity sensor is a weighing sensor with a range of 0-100 kg, installed at a bottom of the hopper of feeding equipment, and connected to the industrial control computer via the RS485 bus after being connected in series with the feeding time recorder; and
the RFID identification unit comprises an RFID reader and an RFID electronic tag; wherein the RFID reader uses a high-frequency reader with a working frequency of 13.56 MHz and a reading distance of 0-10 cm, and the RFID reader is connected to an industrial control computer through a TCP/IP protocol.

3. The whole-process traceability system for aquatic products based on blockchain technology according to claim 2, wherein a specific structure of the feeding monitoring unit is as follows:
the feeding amount sensor adopts a parallel beam structure, a temperature compensation circuit is placed inside, the temperature compensation circuit is fixed at the bottom of the hopper by four bolts, at an inclination angle of not more than 2°; and
the feeding time recorder uses an industrial-grade single-chip microcomputer controller with a real-time clock chip; and a 3.5-inch LCD;
and wherein a 4-core shielded cable is used to connect the feeding quantity sensor and the feeding time recorder.

4. The whole-process traceability system for aquatic products based on blockchain technology according to claim 1, wherein the edge server and industrial control computer are as follows:
the industrial control computer uses a Linux real-time operating system to collect sensor data through a data acquisition program; the data acquisition program comprises a data verification module, a data filtering module, and a data compression module; the data verification module uses a CRC verification algorithm to ensure data integrity; the data filtering module uses a median filtering algorithm to remove outliers, and the data compression module uses a lossless compression algorithm to reduce an amount of data transmission;
the edge server adopts a Docker containerized deployment and runs an edge computing framework, the edge computing framework comprises a data analysis engine, a feature extraction engine and a data fusion engine; wherein the data analysis engine parses collected original data into a standard format, the feature extraction engine uses a sliding time window method to extract data features, and the data fusion engine uses a Kalman filter algorithm to fuse multi-source data; and
the edge server interacts with the blockchain layer through a message queue, and uses a publish or subscribe mode to realize an asynchronous data transmission, wherein the message queue is implemented by RabbitMQ, and a data transmission security is ensured by SSL encryption.

5. The whole-process traceability system for aquatic products based on blockchain technology according to claim 4, wherein a specific implementation architecture of the industrial control computer is as follows:
the data acquisition program is based on a multi-threaded architecture design, comprising a device driver thread, a data acquisition thread, and a data processing thread, wherein:
the device driver thread is responsible for managing a communication interface between an RS-485 device and a TCP/IP device; an epoll mechanism is used to achieve high-concurrent IO processing;
the data acquisition thread adopts a polling mode to sample each sensor at regular intervals, a sampling period can be configured in a range of 100 ms-1000 ms, and a double buffer mechanism is used to avoid data loss;
the data processing thread receives the collected data through a pipeline mechanism, the processed data interacts with an application program through shared memory; and
the industrial control computer also comprises a watchdog program, which is responsible for monitoring a running state of the system; when a system anomaly is detected, a relevant process is automatically restarted, meanwhile, a system log is recorded through a syslog mechanism.

6. The whole-process traceability system for aquatic products based on blockchain technology according to claim 1, wherein the aquaculture management subsystem, processing management subsystem, logistics management subsystem, and sales management subsystem are as follows:
the processing management subsystem and the logistics management subsystem adopt micro-service architectures and are implemented through a Spring Cloud framework; the processing management subsystem comprises a processing procedure tracking module, a quality inspection module and an inventory management module; the logistics management subsystem comprises a transportation monitoring module, a temperature monitoring module and a distribution management module; a data interaction between each module is performed through RESTful API; and
the sales management subsystem integrates a mobile payment interface and a traceability code query function; and the multi-level authorization mechanism is based on the RBAC (role-based access control) model, comprising four privilege levels of system administrator, enterprise administrator, operator, and consumer.

7. The whole-process traceability system for aquatic products based on blockchain technology according to claim 6, wherein a specific implementation architecture of the sales management subsystem is as follows:
a mobile payment interface adopts a unified payment gateway design, comprising a payment gateway layer, a transaction processing layer and a reconciliation and liquidation layer;
a traceability code query function is based on a QR code technology, comprising a QR code generation layer, data coding layer, and analytical verification layer; and
OAuth2.0 authentication adopts a layered design, comprising an authentication service layer; a resource service layer; a user management layer.

8. An implementation method of the whole-process traceability system for aquatic products based on blockchain technology according to claim 1, wherein the method comprises the following steps:
S1, data collection, comprising collecting multi-source heterogeneous data such as water quality parameters, feeding records, and breeding batches in a breeding process automatically through the equipment of the environmental monitoring unit, feeding monitoring unit, and RFID identification unit to realize an automation of data collection, and an automation rate of collection is more than 95%;
S2, edge computing, comprising using the industrial control computer as an edge gateway of data acquisition, performing a preprocessing of checking, filtering, and compression for original collected data; the edge server further performs data cleaning, feature extraction, and multi-source fusion for the data;
S3, uploading to a blockchain network for storage, comprising uploading optimized structured data to the blockchain network for storage through an API interface provided by a smart contract according to a unified data format;
S4, intelligent analysis, combined with big data analysis and artificial intelligence, wherein each business application subsystem calls the blockchain data to perform real-time monitoring, intelligent early warning, and process optimization of production and operation activities; and
S5, information query, wherein management personnel can manage a background through a Web, consumers can enter a product traceability code through mobile terminals to query quality and safety information of product production, processing, warehousing, and transportation.
